(19) United States
(12) Patent Application Publication
(10) Pub. No.: US 2024/0115484 A1
(43) Pub. Date: Apr. 11, 2024

(54) COMPOSITION COMPRISING POLYMER HYALURONIC ACID NANOPARTICLES

(71) Applicant: AMOREPACIFIC CORPORATION, Seoul (KR)

(72) Inventors: Hyoseok AN, Yongin-si (KR); Chaeyeon SONG, Yongin-si (KR); Eun Soo LEE, Yongin-si (KR); Seunghan PARK, Yongin-si (KR)

(73) Assignee: AMOREPACIFIC CORPORATION, Seoul (KR)

(21) Appl. No.: 18/466,100

(22) Filed: Sep. 13, 2023

(30) Foreign Application Priority Data
Sep. 15, 2022 (KR) 10-2022-0116111

(51) Int. Cl. A61K 8/73; A61Q 19/00; A61Q 19/08

(52) U.S. Cl. CPC A61K 8/735; A61Q 19/007; A61Q 19/08; A61K 2800/413

(57) ABSTRACT

Disclosed in this specification is a composition comprising polymer hyaluronic acid nanoparticles. In the present disclosure, the polymer hyaluronic acid is micronized to facilitate skin penetration without depolymerization of the polymer hyaluronic acid, and may be stably comprised in a formulation. In addition, the micronized polymer hyaluronic acid comprised in the composition of the present disclosure penetrates the skin and then is neutralized to pH of the skin and expanded in size again, which can provide excellent benefits for moisturizing the skin and improving skin elasticity.

### CROSS REFERENCE TO RELATED APPLICATION

The present application claims priority to Korean Patent Application No. 10-2022-0116111, filed on Sep. 15, 2022, the entire contents of which is incorporated herein for all purposes by this reference.

### BACKGROUND OF THE INVENTION

### Field of the Invention

The present specification relates to a composition comprising polymer hyaluronic acid nanoparticles.

### Description of the Related Art

Hyaluronic acid (HA) is a straight-chain polysaccharide composed of alternately linked β-D-N-acetylglucosamine and β-D-glucuronic acid, which is distributed in the human body in connective tissues such as subcutaneous tissue and cartilage tissue. The hyaluronic acid has excellent biocompatibility and is known to have beneficial effects on the skin, including increasing skin's moisture retention, maintaining skin elasticity, and improving skin barrier function. However, natural hyaluronic acid is not structurally stable and is easily broken down and eliminated in the human body, thus research has been conducted to stabilize natural hyaluronic acid.

A molecular weight of the hyaluronic acid varies depending on a length of a sugar chain. The hyaluronic acid with a low molecular weight is easy to penetrate the skin, but it has a short duration of stay in the skin and a limited ability to retain water, which leads to problems of less effectiveness on the skin, changes in odor, discoloration, and the like. The hyaluronic acid with a high molecular weight has excellent moisturizing and regenerating properties for the skin, but is difficult to penetrate the skin. Therefore, there has been a limitation in realizing the effect of hyaluronic acid on the skin when applied to actual formulations unless the hyaluronic acid is administered directly into the skin through injections.

### SUMMARY OF THE INVENTION

An object to be achieved by the present disclosure is to provide a composition and method of preparation the same comprising a polymer hyaluronic acid that facilitates skin penetration without depolymerization or cross-linking.

An object to be achieved by the present disclosure is to provide a composition and method of preparation the same comprising a polymer hyaluronic acid that is stable in a formulation.

To achieve the above-mentioned objects, there is provided a composition comprising: a polymer hyaluronic acid or salt particles thereof; and an acidic pH adjuster, in which the acidic pH adjuster is an organic acid or inorganic acid, and in which the polymer hyaluronic acid or salt particles thereof have an average particle diameter of 100 nm or less, and wherein pH of the composition is 3.0 or more but less than 5.0.

In addition, an embodiment of the present disclosure provides a method of preparing the composition described above, the method comprises: micronizing a polymer hyaluronic acid or salt thereof by adding an acidic pH adjuster to a solution that comprises the polymer hyaluronic acid or salt thereof, in which the micronized polymer hyaluronic acid or salt particles thereof have an average particle diameter of 100 nm or less, and pH of the composition is 3.0 or more but less than 5.0.

An embodiment of the present disclosure may provide a composition comprising a polymer hyaluronic acid that facilitates skin penetration without depolymerization or cross-linking by micronizing the polymer hyaluronic acid and that is stably comprised in a formulation. The micronized polymer hyaluronic acid according to an embodiment of the present disclosure penetrates the skin and then is neutralized to pH of the skin and expanded in size again, which can provide excellent benefits for moisturizing the skin and improving skin elasticity.

### BRIEF DESCRIPTION OF THE DRAWINGS

FIG. 1 is a graphic comparing a particle size of a composition (Example 1) according to an embodiment of the present disclosure with that of Comparative Example 1.

FIG. 2 is a graph comparing particle sizes of compositions (Examples 1 to 4) according to an embodiment of the present disclosure with that of Comparative Example 2.

FIG. 3 is a graph illustrating molecular weights of a polymer hyaluronic acid nanoparticle comprised in a composition of Comparative Example 1.

FIG. 4 is a graph illustrating molecular weights of a polymer hyaluronic acid nanoparticle comprised in the composition (Example 1) according to an embodiment of the present disclosure.

FIG. 5 is a view confirming depths of absorption into skin tissue of the composition (Example 1) according to an embodiment of the present disclosure and that of Comparative Example 1.

### DETAILED DESCRIPTION OF THE INVENTION

Hereinafter, exemplary embodiments of the present disclosure will be described in detail with reference to the accompanying drawings.

The embodiments of the present disclosure disclosed herein are illustrated for purposes of description only, and the embodiments of the present disclosure may be practiced in various forms and should not be interpreted as limiting to the embodiments described herein. The present disclosure is subject to various modifications and may have various forms, and the embodiments are not intended to limit the present disclosure to any particular disclosure form, but are to be understood to include all modifications, equivalents, or substitutions that fall within the scope of the spirit and art of the present disclosure. Singular expressions include plural expressions unless clearly described as different meanings in the context. In the present application, the terms “comprises,” “comprising,” “includes,” “including,” “containing,” “has,” “having” or other variations thereof are inclusive and therefore specify the presence of stated features, integers, steps, operations, elements, components, and/or combinations thereof, but do not exclude the presence or addition of one or more other features, integers, steps, operations, elements, components, and/or combinations thereof.

An embodiment of the present disclosure may provide a composition including: a polymer hyaluronic acid or salt particles thereof; and an acidic pH adjuster, in which the acidic pH adjuster is an organic acid or inorganic acid, and in which the polymer hyaluronic acid or salt particles have an average particle diameter of 100 nm or less, and wherein pH of the composition is 3.0 or more but less than 5.0.

The term “hyaluronic acid” in the present specification refers to a negative ionic natural polysaccharide that exists in salt form and is a raw material corresponding to EWG rating 1. The hyaluronic acid is mainly produced and purified by microorganisms and is present in the connective tissues and extracellular matrix of the human body, and is often used as a moisturizer because of a high moisture content due to negative ionic functional groups. A molecular structure of the hyaluronic acid may be represented by Chemical Formula 1 below, and may be comprised in the present specification in the form of a salt of the hyaluronic acid.

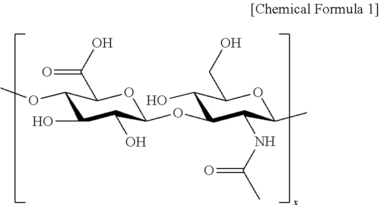

where x is 200 to 20000 mol.

In an embodiment of the present disclosure, the salt may not be specifically limited, but may be acid addition salt, base addition salt, or amino acid salt. For example, the salt may be inorganic salt such as hydrochloride, hydrobromide, sulfate, hydrogen iodide, nitrate, phosphate, etc., organic salt, such as citrate, oxalate, acetate, formate, propionate, benzoate, trifluoroacetate, maleate, tartrate, methanesulfonate, benzenesulfonate, paratoluenesulfonate, etc., inorganic base salt, such as sodium salt, potassium salt, calcium salt, magnesium salt, copper salt, zinc salt, aluminum salt, ammonium salt, etc., organic base salt, such as triethylammonium salt, triethanolammonium salt, pyridinium salt, diisopropylammonium salt, etc., or amino acid salt, such as lysine salt, arginine salt, histidine salt, asparagine salt, glutamine salt, etc. In an embodiment, the salt may be sodium hyaluronate, potassium hyaluronate, hyaluronic acid acetate, hyaluronic acid ammonium salt, or C12-13 alkyl glyceryl hydrolyzed hyaluronate. In the present disclosure, the polymer hyaluronic acid or salt thereof has a weight average molecular weight that varies with a length of a sugar chain, and in an embodiment, the weight average molecular weight of the polymer hyaluronic acid or salt thereof may be 5 to 10,000 kDa. Specifically, the polymer hyaluronic acid or salt thereof may have a weight average molecular weight of 5 kDa or more, 10 kDa or more, 100 kDa or more, 500 kDa or more, 1000 kDa or more, 1,100 kDa or more, 1,200 kDa or more, 1,300 kDa or more, 1,400 kDa or more, 1,500 kDa or more, 1,600 kDa or more, 1,700 kDa or more, 1,800 kDa or more, 1,900 kDa or more, 2,000 kDa or more, 2,200 kDa or more, 2,400 kDa or more, 2,600 kDa or more, 2,800 kDa or more, 2,900 kDa or more, 3,000 kDa or more, 4,000 kDa or more, or 5,000 kDa or more, and may have a weight average molecular weight of 10,000 kDa or less, 9,000 kDa or less, 8,000 kDa or less, 7,000 kDa or less, 6,000 kDa or less, 5,000 kDa or less, 4,500 kDa or less, 4,000 kDa or less, 3,900 kDa or less, 3,800 kDa or less, 3,700 kDa or less, 3,600 kDa or less, 3,500 kDa or less, 3,400 kDa or less, 3,300 kDa or less, 3,200 kDa or less, 3,100 kDa or less, 3,000 kDa or less, 2,500 kDa or less, 2,000 kDa or less, 1,900 kDa or less, 1,800 kDa or less, 1,700 kDa or less, 1,600 kDa or less, 1,500 kDa or less, 1,000 kDa or less, 500 kDa or less, 100 kDa or less, or 10 kDa. More specifically, the weight average molecular weight of the polymer hyaluronic acid or salt thereof may be 1,000 to 5,000 kDa. When the weight average molecular weight of the polymer hyaluronic acid or salt thereof is less than 5 kDa, the moisturizing effect may be reduced, and when the weight average molecular weight is more than 10,000 kDa, the polymer hyaluronic acid or salt thereof may have excessively high viscosity or may have limited micronization, which makes it difficult to penetrate the skin.

In an embodiment of the present disclosure, as polymer hyaluronic acid particles are comprised with the acidic pH adjuster, a net charge in the molecule of negatively charged hyaluronic acid is adjusted by hydrogen cations generated by acid from the acidic pH adjuster while the molecular weight of the polymer hyaluronic acid is maintained. Therefore, the polymer hyaluronic acid particles may be micronized into nano-sized particles. In more detail, as the hydrogen cations of the acidic pH adjuster increase in a solution and neutralize the negative charge of the polymer hyaluronic acid, the charge density of the polymer hyaluronic acid decreases. Therefore, polymer chains of the polymer hyaluronic acid may have a spherical shape instead of being linear in the solution. That is, since radius of gyration (globular size) of the polymer chain is reduced, which may reduce an average diameter of the particles, the probability of hydrogen bonding of hyaluronic acid within the polymer chain increases, which allows micronization to proceed at a smaller size.

Accordingly, the composition according to an embodiment of the present disclosure facilitates the penetration of the polymer hyaluronic acid particles into the skin, resulting in increased skin penetration rate and depth of penetration, and therefore enables delivery of the polymer hyaluronic acid to the lower stratum corneum upon dermal administration. Further, according to an embodiment of the present disclosure, since the micronized polymer hyaluronic acid particles, after penetrating the skin, neutralize in the environment of pH of (about pH 5 to 6) the skin without the need for a separate two-liquid formulation, the size of the polymer hyaluronic acid particles again expands by 200% or more, more specifically, by 200% or more, 210% or more, 250% or more, 300% or more, 350% or more, 400% or more, or 450% or more, the micronized polymer hyaluronic acid particles may provide useful benefits to the skin, such as moisturizing the skin due to the effect of the micronized polymer hyaluronic acid particles swelling themselves, preventing or improving wrinkles in the skin, and preventing or improving the reduction in elasticity of the skin. Further, the composition comprising the micronized polymer hyaluronic acid particles according to an embodiment of the present disclosure maintains a stable formulation even at a high temperature, thereby solving the problem in that the conventional composition comprising polymer hyaluronic acid has broken formulation and become less viscous due to high temperature treatment during an aging process for productization. In an embodiment, the composition may provide excellent penetration into the skin and formulation stability of polymer hyaluronic acid through micronization, without the need for polyhydric alcohols or solubilizing agents.

In an embodiment, the micronized polymer hyaluronic acid particles may have an average particle diameter of 100 nm or less. As used in the present specification, the particle diameter of the particle means the largest diameter in the particle, and an average of the particle diameters means an average of the particle diameters of at least 90% or more of the particles distributed in the composition. Specifically, the average particle diameter of the particles, that is, an average of particle diameters, may mean an average value of the largest diameter in a particle of at least 90% or more, 91% or more, 92% or more, 93% or more, 94% or more, 95% or more, 96% or more, 97% or more, 98% or more, or 99% or more of the particles distributed in the composition. More specifically, the average particle diameter may be greater than 0 nm but 100 nm or less, 95 nm or less, 90 nm or less, 85 nm or less, 80 nm or less, 75 nm or less, 70 nm or less, 65 nm or less, 60 nm or less, 55 nm or less, 50 nm or less, 45 nm or less, 40 nm or less, 35 nm or less, 30 nm or less, 25 nm or less, 20 nm or less, 15 nm or less, or 10 nm or less.

In view of the micronization of polymer hyaluronic acid particles as described above, the pH of the composition according to an embodiment may be 3.0 or more but less than 5.0. Specifically, the pH of the composition may be 3 or more, 3.1 or more, 3.2 or more, 3.3 or more, 3.4 or more, 3.5 or more, 3.6 or more, 3.7 or more, 3.8 or more, 3.9 or more, 4.0 or more, 4.1 or more, 4.2 or more, 4.3 or more, 4.4 or more, 4.5 or more, 4.6 or more, 4.7 or more, 4.8 or more, or 4.9 or more, and may be 5.0 or less, 4.9 or less, 4.8 or less, 4.7 or less, 4.6 or less, 4.6 or less, 4.5 or less, 4.4 or less, 4.3 or less, 4.2 or less, 4.1 or less, 4.0 or less, 3.9 or less, 3.8 or less, 3.7 or less, 3.6 or less, 3.5 or less, 3.4 or less, 3.3 or less, 3.2 or less, or 3.1 or less. When the pH of the composition according to an embodiment is less than 3.0, the composition may be irritating to the skin and is therefore not suitable for formulation into a cosmetic composition according to distribution cosmetic safety control standards. When the pH is 5.0 or greater than 5.0, the polymer hyaluronic acid particles may not be able to be micronized to an average particle diameter of 100 nm or less.

In an embodiment, the acidic pH adjuster may be organic or inorganic acid, and the type thereof is not limited, as long as the acidic pH adjuster is capable of adjusting the pH of the composition to 3.0 or more but less than 5.0. For example, the organic acid may comprise one or more species selected from the group consisting of citric acid, phosphoric acid, fumaric acid, ascorbic acid, ferulic acid, succinic acid, lactic acid, hyaluronic acid, malic acid, butyric acid, formic acid, acetic acid, salicylic acid, glycolic acid, tartaric acid, and gluconolactone. For example, the inorganic acid may comprise one or more species selected from the group consisting of carbonic acid, hydrochloric acid, nitric acid, sulfuric acid, and the like.

In an embodiment, the acidic pH adjuster may comprise a fruit extract comprising the organic acid or inorganic acid described above, and may be used without limitation for the purpose of lowering pH in skincare products, whether or not the acidic pH adjuster is within the scope thereof. For example, the fruit extract may comprise one or more species selected the group consisting of Vaccinium myrtillus fruit extract, Saccharum officinarum (sugar cane) extract, citrus Aurantium dulcis (orange) fruit extract, and Citrus limon (lemon) fruit extract, and the like.

In an embodiment of the present disclosure, the organic or inorganic acid is not comprised in the form of a salt, but is comprised as an acid itself. Unlike, for example, when a metal salt of an organic acid is comprised, where the metal salt acts as a metal cation, a hydrogen cation of the acid may interact with a negative charge of the polymer hyaluronic acid to adjust the pH of the composition to less than 5.0. Accordingly, the polymer hyaluronic acid may be micronized such that the average diameter of the particle is 100 nm or less.

In an embodiment, a weight ratio of the polymer hyaluronic acid or salt thereof to the acidic pH adjuster may be 1:0.01 to 10. Specifically, the acidic pH adjuster may comprise 0.01 parts by weight or more, 0.1 parts by weight or more, 0.2 parts by weight or more, 0.3 parts by weight or more, 0.4 parts by weight or more, 0.5 parts by weight or more, 0.6 parts by weight or more, 0.7 parts by weight or more, 0.8 parts by weight or more, 0.9 parts by weight or more, 1 part by weight or more, 1.1 parts by weight or more, 1.2 parts by weight or more, 1.3 parts by weight or more, 1.4 parts by weight or more, 1.5 parts by weight or more, 1.6 parts by weight or more, 1.7 parts by weight or more, 1.8 parts by weight or more, 1.9 parts by weight or more, 2 parts by weight or more, or 5 parts by weight or more with respect to 1 part by weight of the polymer hyaluronic acid or salt thereof, and may comprise 10 parts by weight or less, 9 parts by weight or less, 8 parts by weight or less, 7 parts by weight or less, 6 parts by weight or less, 5 parts by weight or less, 4 parts by weight or less, 3 parts by weight or less, 2 parts by weight or less, 1.9 parts by weight or less, 1.8 parts by weight or less, 1.7 parts by weight or less, 1.6 parts by weight or less, 1.5 parts by weight or less, 1.4 parts by weight or less, 1.3 parts by weight or less, 1.2 parts by weight or less, 1.1 parts by weight or less, 0.9 parts by weight or less, 0.8 parts by weight or less, 0.7 parts by weight or less, 0.6 parts by weight or less, 0.5 parts by weight or less, or 0.1 parts by weight or less with respect to 1 part by weight of the polymer hyaluronic acid or salt thereof. When the weight ratio of the acidic pH adjuster to the polymer hyaluronic acid or salt thereof is out of the range above, the particles of the polymer hyaluronic acid or salt thereof may not be micronized or the pH may be too low below 3.0, resulting in skin irritation.

In an embodiment, a content of the polymer hyaluronic acid or salt thereof may be 0.1 to 10 wt % with respect to a total weight of the composition. For example, the content of the polymer hyaluronic acid or salt thereof may be 0.1 wt % or more, 0.3 wt % or more, 0.5 wt % or more, 0.6 wt % or more, 0.7 wt % or more, 0.8 wt % or more, 0.9 wt % or more, 1.0 wt % or more, 1.1 wt % or more, 1.2 wt % or more, 1.3 wt % or more, 1.5 wt % or more, 1.7 wt % or more, 2 wt % or more, 3 wt % or more, 4 wt % or more, 5 wt % or more, 6 wt % or more, 7 wt % or more, 8 wt % or more, or 9 wt % or more, and may be 10 wt % or less, 9 wt % or less, 8 wt % or less, 7 wt % or less, 6 wt % or less, 5 wt % or less, 4 wt % or less, 3 wt % or less, 2.5 wt % or less, 2 wt % or less, 1.5 wt % or less, 1.3 wt % or less, 1.2 wt % or less, 1.1 wt % or less, 1 wt % or less, 0.9 wt % or less, or 0.8 wt % or less. When the content is less than 0.1 wt %, the effect on the skin may be negligible, and when the content is greater than 10 wt %, the content may be excessive relative to the acidic pH adjuster, which may prevent the particles of the polymer hyaluronic acid or salt thereof from being micronized, and cause the viscosity to be too high to be prepared into a liquid formulation.

In an embodiment, a content of the acidic pH adjuster may be 0.01 to 50 wt % based on the total weight of the composition. For example, the content of the acidic pH adjuster may be 0.01 wt % or more, 0.05 wt % or more, 0.1 wt % or more, 0.2 wt % or more, 0.3 wt % or more, 0.4 wt % or more, 0.5 wt % or more, 0.6 wt % or more, 0.7 wt % or more, 0.8 wt % or more, 0.9 wt % or more, 1.0 wt % or more, 1.1 wt % or more, 1.2 wt % or more, 1.3 wt % or more, 1.4 wt % or more, 1.5 wt % or more, 2 wt % or more, 3 wt % or more, 4 wt % or more, 5 wt % or more, 10 wt % or more, 15 wt % or more, 20 wt % or more, 25 wt % or more, 30 wt % or more, 35 wt % or more, 40 wt % or more, or 45 wt % or more, and may be 50 wt % or less, 45 wt % or less, 35 wt % or less, 30 wt % or less, 25 wt % or less, 20 wt % or less, 15 wt % or less, 10 wt % or less, 9 wt % or less, 8 wt % or less, 7 wt % or less, 6 wt % or less, 5 wt % or less, 4 wt % or less, 3 wt % or less, 2 wt % or less, 1.5 wt % or less, 1.4 wt % or less, 1.3 wt % or less, 1.2 wt % or less, 1.1 wt % or less, 1 wt % or less, 0.9 wt % or less, 0.8 wt % or less, 0.7 wt % or less, 0.6 wt % or less, 0.5 wt % or less, 0.4 wt % or less, or 0.1 wt % or less. When the content is less than 0.01 wt %, the particles of the polymer hyaluronic acid or salt thereof may not be micronized, and when the content is more than 50 wt %, skin irritation may occur.

In an embodiment, the composition of the present disclosure may further comprise a metal ion sequestering agent. The metal ion sequestering agent, also referred to as a chelating agent, is generally used to bind to and inactivate metallic ions such as calcium, magnesium, or copper that adversely affect the stability or properties of products such as cosmetics. When the organic acid or inorganic acid is used in the form of a metal salt or the like as the acidic pH adjuster for the micronization of the polymer hyaluronic acid particles, it is difficult to penetrate the skin because the polymer hyaluronic acid particles swell in the composition and are not micronized when the composition comprises the metal ion sequestering agent as described above. In contrast, in an embodiment of the present disclosure, since the organic acid or inorganic acid is comprised as the acid itself rather than in the form of a metal salt or the like, it is possible to micronize the polymer hyaluronic acid particles to an average diameter of 100 nm or less because the micronization is not impeded even if the metal ion sequestering agent is further comprised in order to improve the stability and usability of the products. In addition, since the composition according to the present disclosure reduces the pH to less than 5.0 to micronize the polymer hyaluronic acid particles to penetrate the skin, the pH of the polymer hyaluronic acid particles naturally increases to 5.5 to 6.0 and swells due to a difference in acidity from the natural pH of the skin, thereby increasing in particle size to exhibit a moisturizing or elasticity improving effect without the administration of additional ingredients.

In an embodiment, the metal ion sequestering agent is not limited in type, but may comprise, for example, one or more species of an alkaline salt of ethylene-diamine-tetraacetic acid (EDTA) and an alkaline salt of a phosphate-based compound. More specifically, the metal ion sequestering agent may comprise one or more species selected from the group consisting of disodium EDTA (ethylene-diamine-tetraacetic acid), tetrasodium EDTA, trisodium ethylenediamine disuccinate, sodium phosphate, monosodium phosphate, disodium phosphate, trisodium phosphate, sodium tripolyphosphate, sodium hexametaphosphate, sodium metaphosphate, sodium phytate, tetrasodium pyrophosphate, and sodium acid pyrophosphate. In addition, a content of the metal ion sequestering agent according to an embodiment may be 0.01 to 1 wt % based on the total weight of the composition. Specifically, the content of the metal ion sequestering agent may be 0.001 wt % or more, 0.005 wt % or more, 0.01 wt % or more, 0.02 wt % or more, 0.03 wt % or more, 0.04 wt % or more, 0.05 wt % or more, 0.1 wt % or more, 0.5 wt % or more, or 0.9 wt % or more, and may be 1 wt % or less, 0.9 wt % or less, 0.8 wt % or less, 0.7 wt % or less, 0.6 wt % or less, 0.5 wt % or less, 0.4 wt % or less, 0.3 wt % or less, 0.2 wt % or less, 0.1 wt % or less, 0.05 wt % or less, 0.04 wt % or less, 0.03 wt % or less, or 0.01 wt % or less. When the content of the metal ion sequestering agent is less than 0.01 wt %, the chelating effect may be less, and when the content is more than 1 wt %, the negative effect on the skin may occur by chelating even the beneficial metal ions in the skin cells.

In an embodiment, the composition may provide excellent penetration into the human body and formulation stability of polymer hyaluronic acid through micronization, without the need for polyhydric alcohols or solubilizing agents.

In an embodiment, viscosity or hardness of the composition may vary depending on the formulation of the composition. For example, the viscosity may be 0.001 to 50 Pa·s, but is not limited thereto. For example, the hardness may be 10 to 200 N, but is not limited thereto.

The present disclosure may provide a method of preparing the composition according to the embodiments described above, the method comprises: micronizing a polymer hyaluronic acid or salt thereof by adding an acidic pH adjuster to a solution that comprises the polymer hyaluronic acid or salt thereof, in which the micronized polymer hyaluronic acid or salt particles thereof have an average particle diameter of 100 nm or less, and pH of the composition is 3.0 or more but less than 5.0.

As an embodiment, the method may further comprise adding a metal ion sequestering agent to the solution at the micronizing of the polymer hyaluronic acid or salt thereof.

As an embodiment, the method may further comprise dissolving the polymer hyaluronic acid or salt thereof in a solution that comprises one or more of water and an organic solvent prior to the micronizing of the polymer hyaluronic acid or salt thereof.

As an embodiment, the micronizing of the polymer hyaluronic acid or salt thereof may further comprise stirring and dissolving the solution using a magnetic stirrer.

As an embodiment, the method may further comprise aging the composition at a temperature of 30 to 60° C. after the micronizing of the polymer hyaluronic acid or salt thereof. In this case, the aging time according to an embodiment is not limited, but may be, for example, 5 to 20 hours.

In an embodiment, the composition may be for moisturizing the skin. In an embodiment, the composition may be for preventing loss of skin elasticity or improving skin elasticity. In an embodiment, the composition may be for preventing the formation of skin wrinkles or for improving wrinkles. Another embodiment of the present disclosure may provide a method of moisturizing skin, preventing loss of skin elasticity or improving skin elasticity, or preventing the formation of skin wrinkles or improving wrinkles, the method comprises administering an active amount of a composition to the skin of a subject in need thereof, in which the composition comprises: a polymer hyaluronic acid or salt particles thereof; and an acidic pH adjuster, in which the acidic pH adjuster is an organic acid or inorganic acid, the polymer hyaluronic acid or salt particles thereof have an average particle diameter of 100 nm or less, and pH of the composition is 3.0 or more but less than 5.0. Specifically, there may be provided a method of moisturizing skin, preventing loss of skin elasticity or improving skin elasticity, or preventing the formation of skin wrinkles or improving wrinkles, the method comprises: applying a composition to the skin of a subject in need thereof, in which the composition comprises an active amount of polymer hyaluronic acid or salt particles thereof; and an acidic pH adjuster, in which the acidic pH adjuster is an organic acid or inorganic acid, the polymer hyaluronic acid or salt particles thereof have an average particle diameter of 100 nm or less, and pH of the composition is 3.0 or more but less than 5.0. Still another embodiment may provide a use of a polymer hyaluronic acid or salt particles thereof; and an acidic pH adjuster, for preparing a composition for moisturizing skin, preventing loss of skin elasticity, improving skin elasticity, preventing the formation of skin wrinkles, or improving wrinkles. Still another embodiment may provide a polymer hyaluronic acid or salt particles thereof; and an acidic pH adjuster, for using in a composition for moisturizing skin, preventing loss of skin elasticity, improving skin elasticity, preventing the formation of skin wrinkles, or improving wrinkles. Yet another embodiment may provide a non-therapeutic cosmetic use of a polymer hyaluronic acid or salt particles thereof; and an acidic pH adjuster as an active ingredient for preparing a composition for moisturizing skin, preventing loss of skin elasticity, improving skin elasticity, preventing the formation of skin wrinkles, or improving wrinkles. In this case, the acidic pH adjuster is an organic acid or inorganic acid, an average particle diameter of the polymer hyaluronic acid or salt particles thereof is 100 nm or less, and pH of the composition may be 3.0 or more but less than 5.0.

In an embodiment, the composition may be a composition for skin topical agent. As used in the present disclosure, the term “composition for skin topical agent” means a composition of a formulation that is applied externally to the skin. As an embodiment, the composition for skin topical agent may be applied by hand or using various application means, such as a spatula, and the like, and may comprise various formulations of cosmetics, pharmaceuticals, non-pharmaceuticals, and the like.

In an embodiment, the composition may be a cosmetic composition. In an embodiment, an appearance of the cosmetic composition may comprise a cosmetically or dermatologically acceptable medium or carrier. For example, the appearance of the cosmetic composition may be provided in the form of a solution, gel, solid, pasty anhydrous product, emulsion obtained by dispersing an oil phase in an aqueous phase, suspension, microemulsion, microcapsule, microgranule, or ionic (liposome) and non-ionic vesicular dispersions, or in the form of a cream, skin, lotion, powder, ointment, spray, or conceal stick. These compositions may be prepared according to conventional methods in the art. The composition according to the present disclosure may also be used in the form of a foam or in the form of an aerosol composition further comprising a compressed propellant.

The cosmetic composition according to an embodiment of the present disclosure is not particularly limited in the formulation and may be formulated into cosmetic products such as, for example, an ampoule, cream, softening cosmetic water, astringent cosmetic water, nutritional cosmetic water, nutritional cream, gel cream, massage cream, essence, eye cream, eye essence, cleansing cream, cleansing foam, cleansing water, and a cleansing tissue, pack, body lotion, body cream, body oil, and body essence that comprise the cosmetic compositions. When the formulation according to an embodiment of the present disclosure is a paste, cream or gel, an animal fiber, plant fiber, wax, paraffin, starch, tragacanth, cellulose derivative, polyethylene glycol, silicone, bentonite, silica, talc or zinc oxide may be used as a carrier component. When the formulation according to an embodiment of the present disclosure is a spray, a lactose, talc, silica, aluminum hydroxide, calcium silicate, or polyamide powder may be used as a carrier component, and a propellant such as chlorofluorohydrocarbon, propane/butane, or dimethyl ether may be further comprised. When the formulation according to an embodiment of the present disclosure is a solution or emulsion, a solvent, solubilizer or emulsifier is used as a carrier component, for example, there are water, ethanol, isopropanol, ethyl carbonate, ethyl acetate, benzyl alcohol, benzyl benzoate, propylene glycol, 1,3-butylglycol oil, glycerol aliphatic esters, polyethylene glycol or fatty acid esters of sorbitan. When the formulation according to an embodiment of the present disclosure is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum methahydroxide, bentonite, agar or tragacanth may be used as a carrier component. When the formulation according to an embodiment of the present disclosure is a surfactant-containing cleansing, aliphatic alcohol sulfate, aliphatic alcohol ether sulfate, sulfosuccinic acid monoester, isethionate, imidazolinium derivatives, methyl taurate, sarcosinate, fatty acid amide ether sulfate, alkylamidobetaine, aliphatic alcohol, fatty acid glyceride, fatty acid diethanolamide, vegetable oil, linolein derivatives or ethoxylated glycerol fatty acid ester may be used as a carrier component.

The cosmetic composition according to an embodiment of the present disclosure may further comprise functional additives and ingredients comprised in a general cosmetic composition. The functional additives may comprise ingredients selected from the group consisting of a water-soluble vitamin, oil-soluble vitamin, polymer peptide, polymer polysaccharide, sphingolipid, and seaweed extract. In addition, blending ingredients that may be comprised in the cosmetic composition comprise a retaining moisture ingredient, moisturizer, emollient, neutralizer, surfactant, organic and inorganic pigment, organic powder, UV absorber, preservative, bactericide, antioxidant, plant extract, pH adjuster, alcohol, pigment, fragrance, blood flow stimulant, cooling agent, antiperspirant, purified water, and the like.

In an embodiment, the composition may be a pharmaceutical composition. When the composition according to an embodiment of the present disclosure is applied to a pharmaceutical product, the active ingredient used in an embodiment of the present disclosure may be added to an inorganic or organic carrier that is commonly available and formulated into a semi-solid or liquid form of non-oral administration, and the pharmaceutical composition according to the present disclosure may be administered non-orally, rectally, topically, transdermally, intravenously, intramuscularly, intraperitoneally, subcutaneously, and the like. In an embodiment, the pharmaceutical composition may be a non-oral agent, more specifically a skin topical agent. For example, the skin topical agent may be a formulation such as an ointment, skin, lotion, gel, cream, spray, suspension, emulsion, patch, or the like, but is not limited thereto. The composition according to an embodiment of the present disclosure may be prepared according to commonly known methods in the art to facilitate the formulation of the active ingredient, in which case surfactants, excipients, colorants, fragrances, preservatives, stabilizers, buffers, suspending agents, and other commonly available supplements may be appropriately used. A dosage amount of the active ingredient of the pharmaceutical composition according to an embodiment of the present disclosure will depend on an age, sex, weight, pathologic state and severity of a subject to be administered, a route of administration, or judgment of a prescriber. A determination of the appropriate dosage amount based on these factors is within the skill of those skilled in the art and a daily dosage amount may be, for example, 0.1 to 100 mg/kg/day, and more specifically, 5 to 50 mg/kg/day, but is not limited thereto.

In an embodiment, the composition may be a food composition. The food composition according to an embodiment of the present disclosure may be a formulation in a liquid or solid state, and may be a formulation of a tablet, capsule, soft capsule, lozenge, granule, beverage (drink), diet bar, chocolate, caramel formulation, or snack, but is not particularly limited thereto. In addition to the active ingredients above, the food composition according to an embodiment of the present disclosure may comprise excipients, sugars, flavors, colors, oils, proteins, and the like as needed.

Hereinafter, the present disclosure will be described in more detail with reference to the following embodiments. However, the following embodiments are provided for illustrative purposes only to assist in understanding the present disclosure and are not intended to limit the scope and range of the present disclosure.

### Test Example 1

To confirm the micronization of the polymer hyaluronic acid or salt thereof according to the present disclosure, as an embodiment, Examples 1 to 4 having the composition of Table 1 below were prepared by the following method.

First, sodium hyaluronate (manufacturer: Hyundai Bioland, product name: Bio sodium hyaluronate, weight average molecular weight: 1,500 KDa) was prepared as a polymer hyaluronic acid salt. In Examples 1 to 4, 1,2-hexanediol and sodium hyaluronate were dissolved in deionized water using a magnetic stirrer, and then the remaining ingredients were added and mixed with a homogenizer at a high speed of 12000 rpm to induce particle micronization. In this case, the pH of the micronized solution was in the range of 3 to 4. After the hyaluronic acid solution was input into the 45° C. chamber, a final composition was prepared by stabilizing the particles and properties through an aging process for 12 hours or more.

As a comparative example to compare the effects of the examples above, Comparative Example 1, which comprises only a solvent and sodium hyaluronate, and Comparative Example 2, which comprises trisodium citrate (pH 6.5) that is a type of conjugate base, that is, a metal salt of an organic acid, as a pH adjuster, were prepared in the same manner as Examples 1 to 4 above.

TABLE 1

| | | | | | Comparative | Comparative |
|---|---|---|---|---|---|---|
| Ingredients | Example 1 | Example 2 | Example 3 | Example 4 | Example 1 | Example 2 |
| Deionized water | To 100 | To 100 | To 100 | To 100 | To 100 | To 100 |
| 1,2-Hexanediol | 1.2 | 1.2 | 1.2 | 1.2 | 1.2 | 1.2 |
| Sodium | 0.8 | 0.8 | 0.8 | 0.8 | 0.8 | 0.8 |
| Hyaluronate | | | | | | |
| Ascorbic acid | 0.9 | — | — | 0.9 | — | — |
| Succinic acid | — | 1.5 | — | — | — | — |
| Citric Acid | — | — | 0.4 | — | — | — |
| Trisodium citrate | — | — | — | — | — | 0.5 |
| Disodium EDTA | — | — | — | 0.03 | — | — |

(wt %)

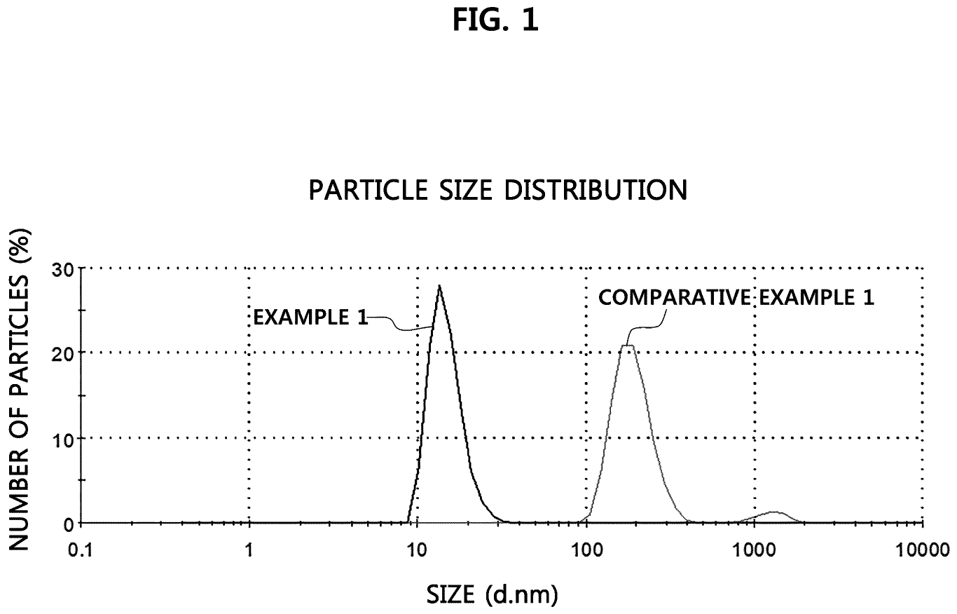

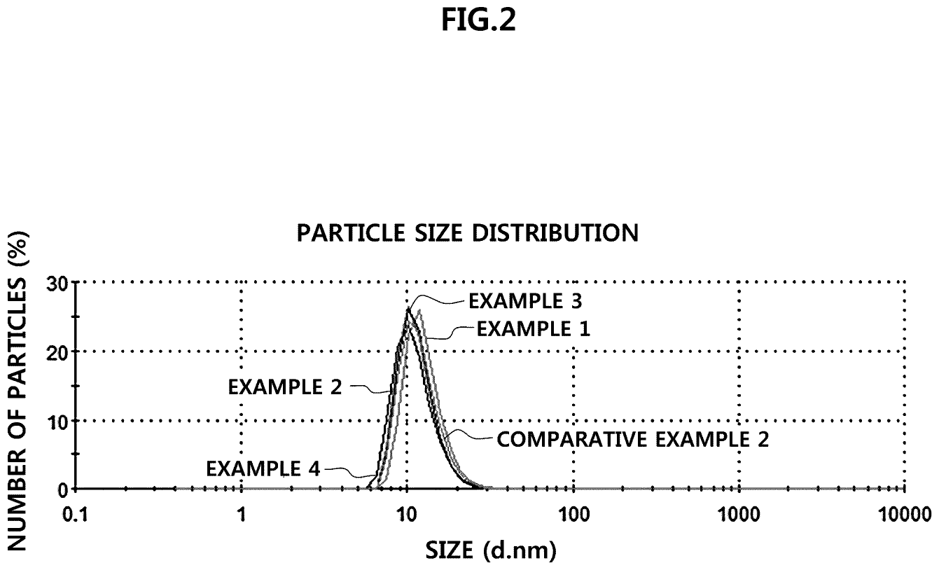

To compare the micronization of the polymer hyaluronic acids or salts thereof of Examples 1 to 4 with that of Comparative Examples 1 and 2, the average particle diameter of each composition was measured using a Zetasizer instrument from Malvern Panalytical company, and is illustrated in FIGS. 1 and 2.

As a result, as illustrated in FIGS. 1 and 2, it can be seen that the particles of the polymer hyaluronic acid salt of Comparative Example 1, which is an aqueous solution of polymer hyaluronic acid without the addition of the pH adjuster, were distributed in the range of 100 nm to 1000 nm and did not decrease in particle size, but the particles of the polymer hyaluronic acid salts of Examples 1 to 4 were all distributed in the range of 10 nm to 100 nm in size even with the change of acid type and the addition of the metal ion sequestering agent, and were micronized to about 10% or less of that of Comparative Example 1. In Comparative Example 2, the average particle size of the polymer hyaluronic acid salt was found to be less than 100 nm, but there is a limitation that the particle size does not expand again after being administered to the skin by the metal salt of the organic acid being used as the pH adjuster, as confirmed in Test Example 2 described below.

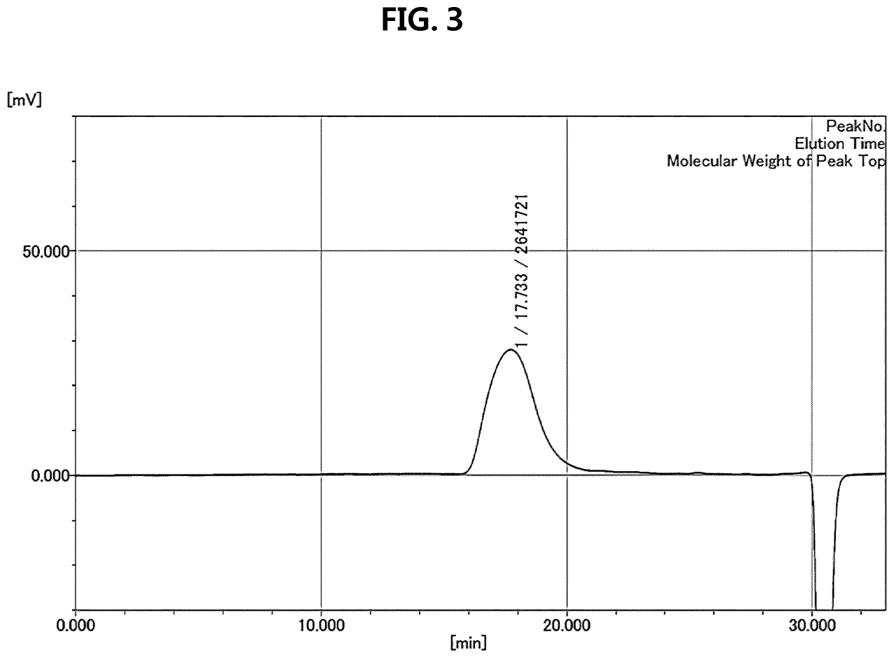

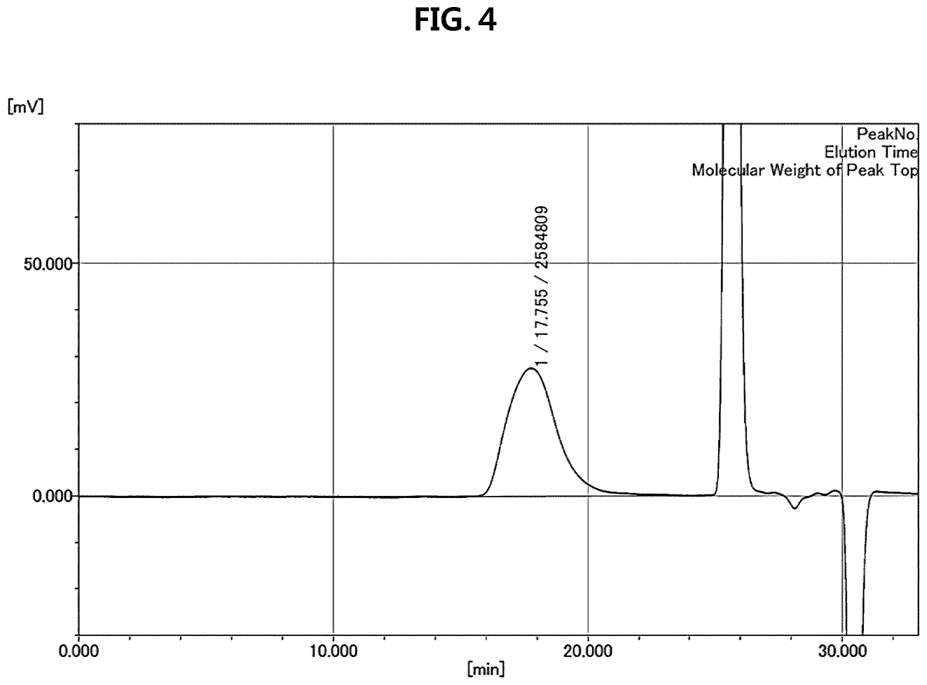

In addition, as a result of measuring relative molecular weights of Example 1 and Comparative Example 1 by gel chromatography (GPC) requested to Korea Polymer Testing Institute (Koptri), an accredited testing agency, it was found that both polymer hyaluronic acid salts of Comparative Example 1 (FIG. 3) and Example 1 (FIG. 4) showed the same peak at 17 minutes, indicating that the maximum molecular weights were the same. The results above mean that in Example 1, the polymer hyaluronic acid salt was micronized to a particle size of 100 nm or less without changing the molecular weight of the polymer hyaluronic acid salt.

### Test Example 2

It was confirmed whether the polymer hyaluronic acid or salt particles thereof that are micronized according to the present disclosure are capable of providing the effect of the polymer hyaluronic acid salt on the skin, such as moisturizing the skin or improving skin elasticity, by the polymer hyaluronic acid or salt particle thereof being administered to the skin, neutralized by the pH of the skin, and then expanded in size again, through a change in the particle size of the micronized polymer hyaluronic acid or salt particle thereof in response to a change in pH, with respect to Examples 1 to 4 prepared in Test Example 1 and Comparative Example 2.

Specifically, a base neutralizer (manufacturer: ANGUS, product name: Tromethamine) was added to each of Examples 1 to 4 and Comparative Example 2 prepared in Test Example 1 above to increase the pH of each composition to pH 6, which is the same as the pH of the skin, and then a change in particle behavior was confirmed using Zetasizer (Malvern, UK) instrument.

As a result, as shown in Table 2 below, it was confirmed that for Examples 1 to 4, the particle size increased again as the pH of the composition increased compared to before the pH increased, but for Comparative Example 2, the change in particle size was insignificant and rather the particle size slightly decreased.

TABLE 2

| | Exam- | Exam- | Exam- | Exam- | Comparative |
|---|---|---|---|---|---|
| | ple 1 | ple 2 | ple 3 | ple 4 | Example 2 |
| Average particle | 460% | 390% | 210% | 250% | −10% |
| diameter increase | | | | | |
| rate (%) | | | | | |

### Test Example 3

A change in physical properties of the composition according to the present disclosure was confirmed by viscosity measurements with respect to Examples 1 to 4 prepared in Test Example 1 and Comparative Example 1.

In this case, viscosity (Pa·s) was measured using a rheometer (Malvern, UK), plate type at 25° C.: oscillation stress adjusted to 1 Pa using CP 4/40, frequency range of 0.1-10 Hz, 1 Pa shear stress, and 1 mm gap are set conditions. Among those, G′ and G″ values at 1 Hz were measured, and the G′ and G″ measurement samples were standardized in the form of a disk with a diameter of 20 mm and a thickness of 1 mm.

As a result, as shown in Table 3 below, it can be seen that for Examples 1 to 4, even though the hyaluronic acid solution was aged at 45° C. for 12 hours or more during preparation, the physical properties of the composition were stabilized at a low viscosity.

TABLE 3

| | Exam- | Exam- | Exam- | Exam- | Comparative |
|---|---|---|---|---|---|
| | ple 1 | ple 2 | ple 3 | ple 4 | Example 1 |
| Viscosity (Pa · s) | 0.023 | 0.025 | 0.018 | 0.022 | 0.306 |

### Test Example 4

To confirm the increase in skin penetration by the micronization of the polymer hyaluronic acid or salt particles thereof according to the present disclosure, the following experiment was conducted.

First, skin tissue of Fitzpatric type II (manufacturer: Genoskin, product name: NativeSkin) biopsied from the abdomen of a 33-year-old female was supplied by the manufacturer, and Example 1 and Comparative Example 1 prepared in Test Example 1 were applied to the skin tissue, respectively. A tape disk (manufacturer: Cuderm, product name: D100, diameter: 22 mm) was attached to the skin tissue, pressed with a D-squame pressure device with a force of 225 gr./sq.cm, and then the tape disk was removed. A total of 10 tape disks were sequentially removed at approximately 3-minute intervals, and the tape disk samples were stored in a freezer.

A desorption electrospray ionization (DESI) mass spectrometer (manufacturer: Waters, product name: 2D DESI stage & XEVO G2-XS QTof) was used to confirm whether each composition was permeable at each sequential order from tape disc samples stored in the freezer. In this case, DESI-MS spectra were acquired using the 2D DESI stage and XEVO G2-XS QTof (Waters, Milford, USA). A spray solvent was 98% methanol+0.1% formic acid, a flow rate was 2 μL/min, and a spray gas inlet pressure was set to 0.5 MPa. An area of 100×100 μm² was measured on the tape-stripped tape disk sample above. A mass range of 50 to 1,000 m/z was measured in a positive ion mode. MS imaging data were acquired using MassLynx 4.2 (Waters, USA) and visualized using High Definition Imaging (HDI) 1.5 software (Waters).

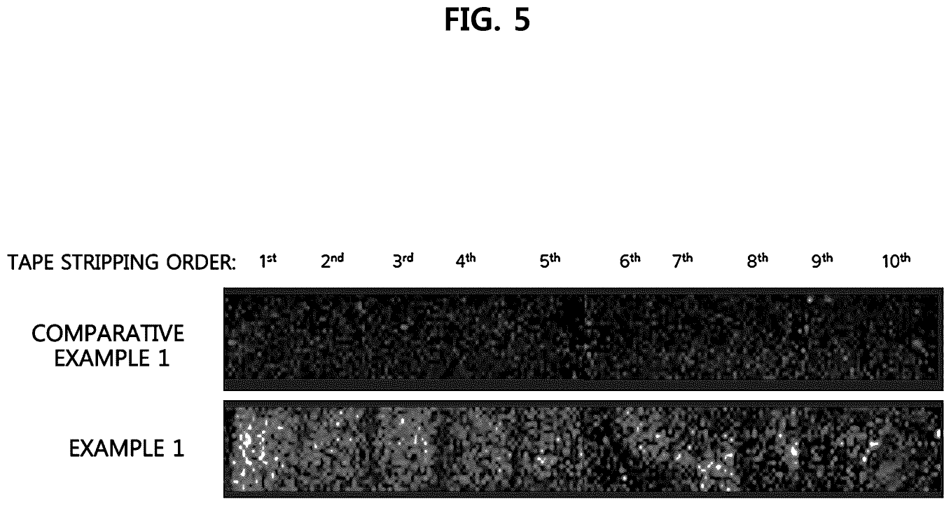

As a result, as illustrated in FIG. 5, for Comparative Example 1, it was not confirmed that any particles penetrated into the stratum corneum due to the large particle size of the polymer hyaluronic acid salt, whereas for Example 1 according to an embodiment of the present disclosure, it was confirmed that there was deep penetration up to the 10th order due to the micronization of the polymer hyaluronic acid salt.

The present disclosure may provide the following examples as an embodiment.

A first example may provide a composition comprising: a polymer hyaluronic acid or salt particles thereof; and an acidic pH adjuster, in which the acidic pH adjuster is an organic acid or inorganic acid, and in which the polymer hyaluronic acid or salt particle thereof have an average particle diameter of 100 nm or less, and wherein pH of the composition is 3.0 or more but less than 5.0.

A second example may provide the composition of the first example, which further comprises a metal ion sequestering agent.

A third example may provide the composition of the first or second example, in which the metal ion sequestering agent is one or more species of an alkaline salt of ethylene-diamine-tetraacetic acid (EDTA) and an alkaline salt of a phosphate-based compound.

A fourth example may provide the composition of any one of the first to third examples, in which the polymer hyaluronic acid or salt thereof has a weight average molecular weight of 5 to 10,000 kDa.

A fifth example may provide the composition of any one of the first to fourth examples, in which a weight ratio of the polymer hyaluronic acid or salt thereof to an acidic pH adjuster is 1:0.01 to 10.

A sixth example may provide the composition of any one of the first to fifth examples, in which the polymer hyaluronic acid or a salt thereof is comprised at 0.1 to 10 wt %, based on a total weight of the composition.

A seventh example may provide the composition of any one of the first to sixth examples, in which the acidic pH adjuster is comprised at 0.01 to 50 wt %, based on the total weight of the composition.

A eighth-first example may provide the composition of any one of the first to seventh examples, in which the organic acid is one or more species selected from the group consisting of citric acid, phosphoric acid, fumaric acid, ascorbic acid, ferulic acid, succinic acid, lactic acid, hyaluronic acid, malic acid, butyric acid, formic acid, acetic acid, salicylic acid, glycolic acid, tartaric acid, and gluconolactone.

An eighth-second example may provide the composition of any one of the first to eighth-first examples, in which the inorganic acid is one or more species selected from the group consisting of carbonic acid, hydrochloric acid, nitric acid, and sulfuric acid.

A ninth example may provide the composition of any one of the first to eighth-two examples, in which the composition is a composition for topical skin application.

A tenth example may provide the composition of any one of the first to ninth examples, in which the composition is for moisturizing the skin.

An eleventh example may provide the composition of any one of the first to tenth examples, in which the composition is for preventing loss of skin elasticity or improving skin elasticity.

A twelfth example may provide the composition of any one of the first to eleventh examples, in which the composition is a cosmetic composition.

A thirteenth example may provide the composition of any one of the first to twelfth examples, in which the composition is a food composition.

A fourteenth example may provide the composition of any one of the first to thirteenth examples, in which the composition is a pharmaceutical composition.

A fifteenth example may provide a method of preparing the composition of any one of the first to fourteenth examples, the method comprises: micronizing a polymer hyaluronic acid or salt thereof by adding an acidic pH adjuster to a solution that comprises the polymer hyaluronic acid or salt thereof, in which the micronized polymer hyaluronic acid or salt particles thereof have an average particle diameter of 100 nm or less, and pH of the composition is 3.0 or more but less than 5.0.

A sixteenth example may provide the method of the fifteenth example, in which the micronizing of the polymer hyaluronic acid or salt thereof further comprise adding a metal ion sequestering agent to the solution.

A seventeenth example may provide a method of moisturizing skin, preventing loss of skin elasticity, improving skin elasticity, preventing the formation of skin wrinkles or improving wrinkles, using the composition of any one of the first to fourteenth examples, in which the composition comprises an active amount of polymer hyaluronic acid or salt particles thereof; and an acidic pH adjuster.

An eighteenth example may provide the method of the seventeenth example, in which the polymer hyaluronic acid or salt thereof has a weight average molecular weight of 5 to 10,000 kDa.

A nineteenth example may provide the method of the seventeenth or eighteenth example, in which a weight ratio of the polymer hyaluronic acid or salt thereof to the acidic pH adjuster is 1:0.01 to 10.

A twentieth example may provide the method of any one of the seventeenth to nineteenth examples, in which the composition further comprises a metal ion sequestering agent.

What is claimed is:

1. A composition comprising:
a polymer hyaluronic acid or salt particles thereof; and
an acidic pH adjuster, wherein the acidic pH adjuster is an organic acid or inorganic acid, and
wherein the polymer hyaluronic acid or salt particles thereof have an average particle diameter of 100 nm or less,
wherein pH of the composition is 3.0 or more but less than 5.0.

2. The composition of claim 1, further comprising a metal ion sequestering agent.

3. The composition of claim 2, wherein the metal ion sequestering agent is one or more species of an alkaline salt of ethylene-diamine-tetraacetic acid (EDTA) and an alkaline salt of a phosphate-based compound.

4. The composition of claim 1, wherein a weight average molecular weight of the polymer hyaluronic acid or salt thereof is 5 to 10,000 kDa.

5. The composition of claim 1, wherein a weight ratio of the polymer hyaluronic acid or salt thereof to the acidic pH adjuster is 1:0.01 to 10.

6. The composition of claim 1, wherein the polymer hyaluronic acid or salt thereof is comprised at 0.1 to 10 wt % based on a total weight of the composition.

7. The composition of claim 1, wherein the acidic pH adjuster is comprised at 0.01 to 50 wt % based on a total weight of the composition.

8. The composition of claim 1, wherein the organic acid is one or more species selected from the group consisting of citric acid, phosphoric acid, fumaric acid, ascorbic acid, ferulic acid, succinic acid, lactic acid, hyaluronic acid, malic acid, butyric acid, formic acid, acetic acid, salicylic acid, glycolic acid, tartaric acid, and gluconolactone, and
wherein the inorganic acid is one or more species selected from the group consisting of carbonic acid, hydrochloric acid, nitric acid, and sulfuric acid.

9. The composition of claim 1, wherein the composition is a composition for topical skin application.

10. The composition of claim 1, wherein the composition is a cosmetic composition.

11. The composition of claim 1, wherein the composition is a food composition.

12. The composition of claim 1, wherein the composition is a pharmaceutical composition.

13. A method of preparing the composition of claim 1, the method comprising:
micronizing a polymer hyaluronic acid or salt thereof by adding an acidic pH adjuster to a solution that comprises the polymer hyaluronic acid or salt thereof,
wherein the micronized polymer hyaluronic acid or salt particles thereof have an average particle diameter of 100 nm or less, and pH of the composition is 3.0 or more but less than 5.0.

14. The method of claim 13, wherein the micronizing of the polymer hyaluronic acid or salt thereof further comprises adding a metal ion sequestering agent to the solution.

15. The method of claim 13, wherein a weight average molecular weight of the polymer hyaluronic acid or salt thereof is 5 to 10,000 kDa.

16. The method of claim 13, wherein a weight ratio of the polymer hyaluronic acid or salt thereof to the acidic pH adjuster is 1:0.01 to 10.

17. A method of moisturizing skin, preventing loss of skin elasticity or improving skin elasticity, preventing the formation of skin wrinkles, or improving wrinkles, the method comprising:
applying the composition of claim 1 to skin of a subject in need thereof,
wherein the composition comprises:
an active amount of polymer hyaluronic acid or salt particles thereof; and
an acidic pH adjuster.

18. The method of claim 17, wherein a weight average molecular weight of the polymer hyaluronic acid or salt thereof is 5 to 10,000 kDa.

19. The method of claim 17, wherein a weight ratio of the polymer hyaluronic acid or salt thereof to the acidic pH adjuster is 1:0.01 to 10.

20. The method of claim 17, wherein the composition further comprises a metal ion sequestering agent.
